(19) United States
(12) Patent Application Publication
(10) Pub. No.: US 2005/0053565 A1
(43) Pub. Date: Mar. 10, 2005

(54) Modulation of hair growth via SGK3

(76) Inventors: Florian Lang, Tuebingen (DE); David Pearce, San Francisco, CA (US); Jian Wang, Danville, CA (US); James McCormick, San Francisco, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/658,211

(22) Filed: Sep. 9, 2003

(51) Int. Cl. G01N 33/53; A61K 7/06; A61K 7/11

(52) U.S. Cl. 424/70.14; 435/7.1

(57) ABSTRACT

The present invention relates to methods for identifying a hair growth modulating substance, to methods for preparing a pharmaceutical/cosmetic composition for treatment of hair growth disorders, to methods for treating a human being affected by a hair growth disorder, to hair growth modulating substances, to compositions comprising a substance which modulates the activity of SGK3 or causes a modulation of the activity of a SGK3-derived peptide in the above-mentioned “identifying” methods, and to a transgenic non-human sgk3^{-/-} animal for investigating hair growth disorders.

### BACKGROUND OF THE INVENTION

1. Field of the Invention

The present invention is concerned with methods for identifying a hair growth modulating substance, with methods for preparing a pharmaceutical/cosmetic composition for treatment of hair growth disorders, with methods for treating a human being affected by a hair growth disorder, with hair growth modulating substances, and with a transgenic non-human sgk3^{-/-} animal for investigating hair growth disorders.

2. Related Art

Methods of these kinds and hair growth modulating substances are generally known in the art.

Diseases concerning hair growth are a widespread and serious problem by which a large part of the population is affected. Both, hair loss (alopecia) as well as excessive unwanted hair growth (hypertrichosis) have to be regarded in this connection.

Androgenetic alopecia (alopecia androgenetica) is the most frequent form of hair loss. In over 90 percent of the cases, this form is the cause of hair loss with men. Androgenetic alopecia is characterized by baldness caused by miniaturization of genetically predisposed follicles in the male pattern, i.e. frontal recession and thinning at the vertex, or the female pattern, i.e. loss of hair primarily over the crown, with sparing off frontal hair.

So far this kind of hair loss is treated by administering a pharmaceutic or cosmetic composition, for example Finasterid (e.g. Proscar®). Finasterid is a competitive inhibitor of the type-II-5-α-reductase, and has to be administered systemically. In several studies only moderate success of this drug could be shown. Moreover, this moderate success was only observed with male patients but not with female patients. Because of its teratogenic effect Finasterid is contraindicated for use with female patients, since there is the danger of an illness on hypospadia. Nevertheless, a first study on postmenopausal women revealed Finasterid as being ineffective with female patients.

Up to now, female patients affected by androgenetic alopecia have been treated by administering a cyproteron acetate (e.g. Androcour®), which is an anti-androgenic substance. Several studies have revealed moderate success but also severe side effects. Moreover, the application of anti-androgenic substances involves many well-known risks, namely cardiovascularic risks, risks of cancer, and the risk of hyperlipemia.

Another treatment of hair loss in general consists in the topical application of Minoxidil (e.g. Regaine®), which is a potassium channel activator. The application of this substance is also disadvantageous because of its insignificant effectiveness, the appearance of skin irritations or the development of contact allergy and also the occurrence of systemic side effects, such as headache or cardiovascular effects.

Other hair growth disorders concern for example alopecia areata or alopecia atrophicans. The first of those diseases is characterized by a hair loss in patches, thought to be caused by an autoimmune response to hair follicles in a particular stage of hair follicle development. An extensive form of this disorder is called alopecia areata universalis (hair loss over the entire body).

Alopecia atrophicans is characterized by an irreversible destruction of biologically important anatomical hair follicle structures or of the entire hair follicle.

For both diseases no reliable and side effect-free therapies do exist. Partly, corticosteroids are given, whose side effects are well-known in the art.

Another measure for treatment of hair loss is hair transplantation. This measure has several disadvantages. Such transplantations require very painful operations. More-over, these operations are very expensive and not feasible for all kinds of hair growth disorders.

Other serious syndromes concern hypertrichosis which is characterized by excessive and usually diffuse hair growth beyond that considered normal according to age, race, sex, and skin region, as well as hirsutism, i.e. an excessive hair growth in androgen-dependent areas in women.

For treatment of these disorders, normally a drug called Eflornithin (e.g. Vaniqar®) is administered, either topically or systemically, which drug is an inhibitor of the enzyme ornithine decarboxylase that is located within the hair follicle.

Also, this medicament is characterized by an insignificant efficiency. Therefore, the common treatment still consists in shaving the affected areas or by the usage of waxes or depilation crèmes. Unfortunately, these methods are not long-lasting and could have an irritative effect.

Another measure for removing unwanted hair is laser-assisted hair removal. These treatments are very expensive due to high costs of the equipment required.

An overview of the state of the art concerning the therapy of hair growth disorders is given in Trüeb, R. M., “Neues und Bewährtes in der Therapie von Haarerkrankungen”, Der Hautarzt (2003).

So far, there are no approaches for a causal treatment of hair growth disorders, i.e. on a biochemical-physiological or genetic level. The reasons for this are partly founded in the lack of a thorough understanding of the molecular mechanisms of hair growth and hair follicle development; cf. Irvine, A. D. and Christiano, A. M., “Hair on a gene string: recent advances in understanding the molecular genetics of hair loss”, Clinical and Experimental Dermatology (2001) 26, 59-71.

### SUMMARY OF THE INVENTION

In view of the above, an object underlying the present invention is to provide a method for identifying substances which are capable to modulate hair growth in a causal way. Especially, substances should be identified by such a method, which are highly specific and produce less side effects.

According to the invention this object is achieved by a method for identifying a hair growth modulating substance comprising the steps of: (a) contacting of a peptide derived from SGK3 to a test substance under conditions allowing the binding of said test substance to said peptide, and (b) determination, whether said test substance modulates the activity of said peptide.

By this method the object underlying the invention is completely achieved.

Namely, the current inventors have recognized that the SGK3 gene product is involved in hair follicle development and is, therefore, a key target for the modulation of hair growth. They further have realized that this modulation of the hair growth is in direct correlation with the modulation of the activity of SGK3.

Considering the biochemistry of enzymatic active proteins, like SGK3, the inventors have concluded that for modulating hair growth it is sufficient to modulate the activity of that peptide segment that is at least jointly responsible for the enzymatic or catalytic activity of SGK3. Therefore, a substance which modulates the activity of a SGK3-derived peptide, i.e. of a peptide bearing the enzymatic or catalytic activity of SGK3, is capable of modulating hair growth.

In the course of this application, SGK3 stands for the gene product, e.g. for the protein or peptide, whereas sgk3 stands for the genetic information, e.g. for the gene, coding for the gene product.

SGK3 is the serum and glucocorticoid-regulated kinase 3 which is a serine-threonine kinase. Three SGK isoforms have been identified in mammalian cells, which share 75 percent identity in their kinase domains, and are closely related to the Akt kinases. SGK3 (also termed CISK and SGKL) was cloned by homology with SGK1, cf. Dai, F. et al., Genomics (1999) 62, 95-97, and Kobayashi, T. et al., Biochem J (1999) 344 Pt. 1, 189-197, and independently by expression cloning as a factor that promoted IL-3-independent growth in cultured hematopoietic cells, cf. Liu, X. et al., Curr Biol (2000) 10, 1233-1236.

SGK3 is expressed in a wide variety of tissues and shares phosphorylation targets with other members of the SGK/Akt family in vitro.

Uniquely within the SGK/Akt family, SGK3 bears an amino-terminal Phox homology domain, which targets SGK3 to endosomal membranes via its interaction with polyphosoinositides, where it has been co-localized with epidermal growth factor receptor (EGFR).

Like the Akts, the SGKs are all phosphorylated and activated by the phosphoinositide 3-kinase (PI 3-kinase) effector phosphoinositide-dependent kinase-1 (PDK 1) in vitro, and insulin, insulin-like growth factor I, interleukin 3 and EGF activate SGK3 in vivo.

One is assuming that SGK3 is implicated in the control of cell survival and ion channel activity.

So far, a connection between SGK3 and the control of the hair growth has not been recognized.

According to the new method, the test substances have to bind to the SGK3-derived peptide, i.e. a state has to be established in which the substance to be tested is at least in the immediate vicinity of the peptide, and therefore is possibly capable of influencing the activity of the peptide.

“Modulating the activity” means that the peptide is somehow altered in its biochemical/physiological function, whether it is increased or decreased in its activity.

The determination in step (b) is performed by the observation of a binding of the test substance to the peptide, if applicable additionally by the observation of an alteration in the peptide's activity by means of a well-established activity assay.

The substance to be tested can be present in any chemical, biochemical, or biological form conceivable, i.e. as a molecule, like a chemical defined compound or a peptide, protein, antibody, aptamer or as an ion or an atom.

Conditions allowing the binding of the test substance to the peptide are well-known in the area of protein or enzyme biochemistry; those conditions can be provided, for example by the usage of common physiological or biological buffer systems like Tris-, HEPES-, PBS-based buffers, if applicable, supplemented with various kinds of salts in appropriate concentrations as well as with other conventional additives.

The current inventors have found that mice lacking SGK3, so-called sgk3 null or sgk3^{-/-} mice, display an unexpected abnormality in hair growth, i.e. in hair follicle development. These findings of the inventors were completely unforeseen and surprising. So far, SGK3 always has been described in the completely different connections mentioned above.

According to a further object it is determined in step (b) whether the test substance modulates the kinase activity of the peptide.

This measure has the advantage that herewith an alteration of the actual physiological activity of SGK3 is determined, A potential alteration of the kinase activity of the SGK3-derived peptide can be detected by means of well-established kinase activity assays. With such a test it is examined to what degree a model substrate, for example histone HI, GSK3-beta, FKHRL1 and others, will become phosphorylated by the SGK3-derived peptide, before and after its binding to the test substance. The phosphorylation reaction can be followed by the usage of radioactive labeled phosphate, such as ³²P, and by the aid of autoradiographic techniques.

According to another object it is determined in step (b) whether the test substance inhibits or activates the kinase activity of the SGK3-derived peptide.

By this measure, substances can be identified and provided having the potential for down- or up-regulating the activity of SGK3. As a result, substances can be identified which present auspicious therapeutic characteristics for treating hair growth disorders caused by increased or decreased activity of SGK3.

On account of this therapeutic potential of substances identified by the above-mentioned method, another object of the invention relates to a method for preparing a pharmaceutical/cosmetic composition for treatment of hair growth disorders, comprising the steps of: (a) providing a hair growth modulating substance, and (b) formulating said substance into a pharmaceutically/cosmetically acceptable carrier, wherein step (a) is performed by means of the afore-mentioned method for identifying such a substance.

A still further object of the invention relates to a method for treating a human being affected by a hair growth disorder, said method comprising the step of administering a hair growth modulating substance, wherein said substance has been identified by means of the afore-mentioned method.

A pharmaceutical composition, contrary to a cosmetic composition, refers to a composition, which has to be registered as a medicament according to national regulations.

A composition according to the invention can be provided for external application (e.g. as an ointment, creme, liquid, tincture, etc.) as well as for internal application (as tablet, capsule, injection, etc.).

Applicable pharmaceutically and/or cosmetically acceptable carriers are well-known in the art; cf. Kibbe, A. H., “Handbook of Pharmaceutical Excipients”, Third Edition (2000), American Pharmaceutical Association and Pharmaceutical Press, the content of this handbook is hereby incorporated by reference.

Another object of the invention relates to a method for treating a human being affected by a hair growth disorder, comprising the step of administering a hair growth modulating substance, wherein said substance has been identified by means of the afore-mentioned method.

This method for the first time enables a causal treatment of a human being suffering from a hair growth disorder. It shall be understood, that the substance can also be identified by a preferred embodiment of the “identifying”-method according to the invention. The administration of that substance can be performed via topical application, a systemically, given dose, i.e. in form of a tablet, a liquid or an injection, etc.

Against this background another object of the invention relates to a hair growth modulating substance identified by means of the afore-mentioned method, whereby it is preferred if that substance inhibits or activates the activity of the SGK3-derived peptide; as well as to a composition, preferably a pharmaceutical or cosmetic composition, comprising a substance which modulates the activity of SGK3. A further object of the invention relates to a composition, preferably a pharmaceutical or cosmetic composition, comprising a substance which causes a modulation of the activity of a SGK3-derived peptide in the afore-mentioned “identifying”-method.

As mentioned above, these substances can be presented in any possible chemical, biochemical or biological form. Due to the provision of such a substance it is now possible for the first time to treat or even cure several kinds of hair growth disorders, since this substance interacts with a key protein for hair follicle development.

A further object of the invention relates to a method for identifying a hair growth modulating substance, comprising the steps of: (a) providing a transgenic non-human sgk3^{-/-} animal; (b) administering of a test substance to said animal, and (c) determination, whether said test substance modulates hair growth of said animal.

The inventors have thereby provided a testing system for analyzing an unlimited number of potential hair growth modulating substances for their ability, to causally influence hair growth in a reliable manner. To be precise, with the aid of this method substances can be detected which effect hair growth by bypassing the SGK3-pathway.

In this connection, transgenic sgk3^{-/-} animal stands for a sgk3 null or a so-called knockout coat bearing animal, i.e. for an animal without any genetic information coding for a functioning SGK protein. Normally, these animals are characterized by two coding regions of sgk3, i.e. each for every chromosome complement, provided that they are diploid animals. Those knockout animals can be engineered by well-known methods of genetic recombination technologies.

The transgenic non-human animal preferably is a mouse.

This measure has the advantage that for this kind of animals a plurality of reliable methods concerning the knockout of genes have been described in the art.

A further advantage consists in the fact that the genetic organization of the mouse is comparable to those of humans.

In connection with that afore-mentioned method, it is preferred if the substance to be tested is either applied onto the skin of the animal or is applied systemically

By this measure, the administration of a potentially effective substance is performed on the same way, as with a human patient. Thus, also such substances can be tested or identified which can be used as active substances solved within shampoos, hair tonics and the like. Furthermore, substances can be identified which are applicable as active substances in systemically administrable medicines.

It is also preferred if the administration is performed by local injections around and in the affected area(s).

This measure has the advantage that the administration happens in a goal-oriented fashion, by which only areas of hair growth disorders will be treated without affecting areas which are not involved. By this measure side effects are further reduced.

Due to the potential of a substance identified by aid of the preceding method, another object of the invention relates to a method for preparing a pharmaceutical/cosmetic composition for treatment of hair growth disorders, comprising the steps of: (a) providing a hair growth modulating substance, and (b) formulating said substance into a pharmaceutically/cosmetically acceptable carrier, wherein step (a) is performed by means of the afore-mentioned method concerning the usage of the transgenic non-human sgk3^{-/-} animal,

Therefore, a further subject matter also relates to a method for treating a human being affected by a hair growth disorder comprising the step of administering a hair growth modulating substance, wherein said substance has been identified by means of the afore-mentioned method concerning the usage of the transgenic non-human sgk3^{-/-} animal.

Another object of the invention relates to a hair growth modulating substance identified by means of the afore-mentioned method concerning the usage of the transgenic non-human sgk3^{-/-} animal, where it is preferred, if that substance inhibits or activates the activity of the SGK3-derived peptide; as well as to a composition, preferably a pharmaceutical or cosmetic composition, comprising a substance which causes a modulation of the activity of a SGK3-derived peptide in the afore-mentioned method concerning the usage of the transgenic non-human sgk3^{-/-} animal.

Such a transgenic animal can be used as test model or as test system for screening and identifying new hair growth modulating substances which bypass the SGK3-pathway, or for assessing the corresponding potency of hair-care products.

Another object of the present invention relates to a method for treating a human being affected by a hair growth disorder, comprising the steps of: (a) providing a genetic construct coding for an antisense-sgk3 probe and/or for a sgk3-RNAi and/or for a transdominant inhibitory SGK3, and (b) introducing said construct into a human being by means of gene therapeutic methods.

In the alternative, step (a) can be performed by providing a genetic construct comprising a region coding for a sgk3-derived segment under control of a promoter, whereby that promoter preferably is an inducible promoter.

By means of these methods, hair growth disorders can be treated directly on a genetic level, in the first case via so-called antisense-technology, in the second case via classic methods of genetic recombination technology.

An antisense-sgk3 probe is a genetic construct, i.e. a DNA or RNA sequence, which is complementary to a functional messenger RNA or DNA or to parts thereof, coding for SGK3. This probe is capable of annealing to its complementary structure, thereby blocking translation and/or transcription of that coding region.
sgk3-RNAi stands for sgk3-RNA interference, also called siRNA (for “silencing” RNA), and refers to the introduction of homologous double stranded RNA (dsRNA) to specifically target the RNA coding for sgk3. This measure results in a sgk3 null phenotype. This technique was discovered by Fire et al., “Potent and specific genetic interference by double-stranded RNA in Caenorhabditis elegans”, Nature (1998) 391, 806-811. It is the presence of dsRNA, formed from the annealing of sense and antisense strands, that is responsible for producing the interfering activity; the above publication is herewith incorporated by reference. Although the mechanism of how the dsRNA results in the loss of the targeted homologous mRNA is still not well understood, a number of observations indicate that the primary interference effects are post-transcriptional. Because RNAi is remarkably potent (i.e., only a few dsRNA molecules per cell are required to produce effective interference), the dsRNA must be either replicated and/or work catalytically. The current model favors a catalytic mechanism by which the dsRNA unwinds slightly, allowing the antisense strand to base pair with a short region of the target endogenous message and marking it for destruction. “Marking” mechanisms could involve covalent modification of the target, i.e. of the mRNA coding for sgk3 (e.g. by adenosine deaminase) or any number of other mechanisms. Potentially, a single sgk3-RNAi molecule could mark hundreds of target sgk3-mRNAs for destruction before it itself is “spent”.

A transdominant inhibitory SGK3 kinase is characterized by a mutated catalytic subunit, e.g. via a point mutation, and which is therefore not able to display any or sufficient kinase activity. When expressing such a mutant form of SGK3 the wild type SGK3 is suppressed to impalpable background activity.

By the collective term of gene-therapeutic methods according to the invention, those methods have to be understood which cause advantageous changes of a phenotype because of modification or normalization of defect genetic material. Those changes are caused by genes being transfected within cells of a specific tissue for there being expressed or to prevent an erroneous or an unregulated expression of a gene.

The afore-mentioned genetic construct can be introduced into a human being by injection in form of “naked” DNA/cDNA or RNA, plasmids or vectors, possibly by the usage of modificated viruses or transformed bacteria. The genetic construct can also be provided as being included into liposomes, which could be injected or inhaled. In the art, also a gene transfer method is described by which the genetic construct is adsorbed to minute gold particles, which become “shot” into the cells (biolistic method).

Another gene-therapeutic method relates to the so-called ligand-coupled gene transfer. Here, the DNA is coupled to a ligand specific for the target tissue and, therefore, will only be transported into such cells, which carry the appropriate receptor.

With an inducible promoter the activity of that promoter and consequently the expression of sgk3 can be controlled by the state of the promoter. Such a promoter can be activated, i.e. induced, for example via addition of chemicals, tissue specific substances, the pH-value, stress factors, etc. That is why an inducible promoter can also be tissue specific and/or temporary activated, since it will become activated by a substance prevailing exclusively in a particular tissue. Inducible expression systems are described, e.g., in Sambrook J. and Russell D. W., “Molecular Cloning—A Laboratory Manual”, 2001 by Cold Spring Harbor Laboratory Press, which is herewith incorporated in this application by reference.

In view of the above, it is preferred if the genetic construct is selected from the group consisting of: naked DNA or cDNA, naked RNA, plasmid DNA, plasmid RNA, vector DNA, vector RNA, non-virulent/non-pathogenic virus, transformed bacteria.

This measure has the advantage that the genetic construct will be provided in a form for direct usage in established gene transfer methods.

Due to the potential of the antisense technology, as discussed before, a further object of the present invention is a method for preparing a pharmaceutical composition for treatment of hair growth disorders, comprising the steps of: (a) providing a genetic construct coding for antisense-sgk3, and (b) formulating said construct into a pharmaceutically/cosmetically acceptable carrier.

The hair growth disorder addressed in the course of this application is preferably selected from the group consisting of hair loss, preferably induced by chemotherapy; baldness, preferably male pattern baldness; unwanted hair growth, hypertrichosis, hirsutism; alopecia androgenetica; alopecia areata; alopecia areata universalis; alopecia atrophicans.

The advantage of this measure is that some of the most important hair growth disorders are covered, for which so far no goal-orientated causally active substances or causal therapies have been available.

Other advantages ensue from the description and the attached figures.

It will be understood that the features which are mentioned above, and those which are still to be explained below, can use not only in the combinations which are in each case indicated but also in other combinations, or on their own, without departing from the scope of the present invention.

### BRIEF DESCRIPTION OF THE FIGURES

The invention is now explained with the aid of embodiments and the enclosed figures, in which:

FIG. 1 shows the targeting strategy to delete the sgk3 gene;

FIG. 2 shows the result of targeted disruption of the sgk3 allele via (A) genotyping of mice by PCR, (B) Northern blot analysis and (C) Western blot analysis;

FIG. 3 shows the phenotypical changes of mice after targeted disruption of the sgk3 allele;

FIG. 4 and FIG. 5 show the results of histological studies on hair follicle morphogenesis of sgk3 null mice;

FIG. 6 shows the growth development of sgk3 null mice; and

FIG. 7 shows the glucose tolerance of sgk3 null mice.

### DESCRIPTION OF PREFERRED EMBODIMENTS

### EXAMPLE 1

### Construction of Targeting Vector and Generation of sgk3 Null Mice

The targeting strategy for disruption of the sgk3 gene involved removing parts of exons 9 (which contains the ATP-binding site necessary for the catalytic activity of SGK3) and 10, deleting intron 9, and introducing an in-frame STOP codon into exon 10.

Plasmid pNTK loxp was used to generate the targeting vector. Two mouse genomic fragments, containing exons 7-10 and exons 9-16, were amplified from 129Xl/SvJ DNA by PCR and cloned into pCR4-TOPO and pCR-XL-TOPO (both Invitrogen) respectively and characterized by restriction enzymes. The exon 7-10-containing construct was used as a template in a second round of PCR to generate a 2.6-kb exon 7-9 fragment with a BamHI site added to the 5′ end and an MfeI site added to the 3′ end. This fragment was used as the short arm and was inserted into the BamHI/MfeI sites of the targeting vector. The 10-kb long arm fragment was generated by using the exon 9-16-containing construct as a template in a second round of PCR. A ClaI site was added to the 5′ end, and a STOP codon was added before the start of exon 10; a XhoI site was added to the 3′ end. This fragment was inserted into the ClaI/XhoI sites of the targeting vector.

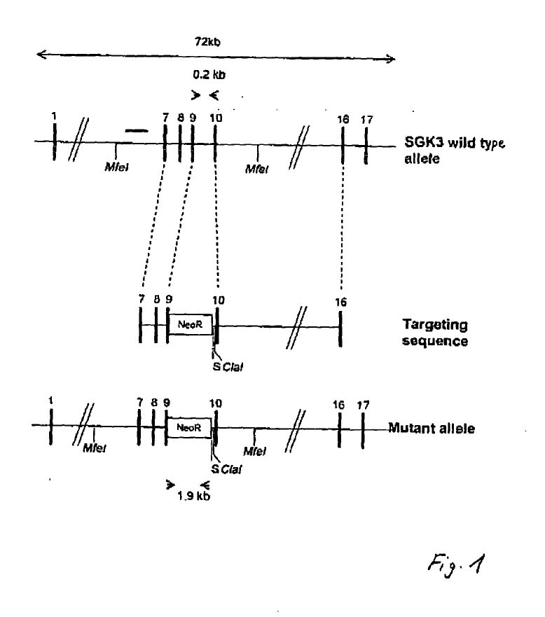

This targeting strategy is shown in FIG. 1. S represents the introduced inframe STOP codon. The locations of PCR primers used in genotyping are indicated by small arrows; the primers marked on the wild type and mutant alleles are identical, but the PCR product size differs, as indicated. The short bar between exons 1 and 7 indicates the DNA fragment used as a DNA probe for Southern blots.

The targeting vector was linearized by digestion with XhoI, and electroporated into RW-4 embryonic stem cells (derived from 129Xl/SvJ mice). G418- and gancyclovir-resistant clones were initially screened by PCR using oligonucleotide primers located inside and outside the targeted locus to confirm homologous recombination. Two positive clones were expanded and their genomic DNA analyzed by Southern blot analysis following digestion by MfeI. An external probe (a 2 kb restriction fragment lying between exons 1 and 7) was used to verify correct targeting.

The two positive clones were injected into C57BL/6 blastocysts and transferred into pseudopregnant females. Chimeric males, identified by their agouti coat color, were mated with C57BL/6 females. To generate mice homozygous for the targeted allele, the resulting sgk3^{+/-} males and females were interbred.

### EXAMPLE 2

### PCR Analysis of Mice Genotype and Sex

Genomic DNA was prepared from tail biopsies by overnight digestion in 500 μl Proteinase K (0.5 mg/ml). Digests were diluted 1:100 and used directly in PCR reactions using primers 5′CTTCT-TGCAAAACGGAAACTGGATG3′ and 5′CCCCTCCATTAACAAAATCCAGAAC3′. PCR products were resolved on 1% agarose gels. The wild type allele PCR product was 0.2-kb; that for the mutated allel was 1.9-kb. Sexing of newborn mice was performed by PCR as described in P. J. McClive, A. E. Sinclair, Mol Reprod Dev 60, 225-6 (2001), whose content is herewith incorporated by reference.

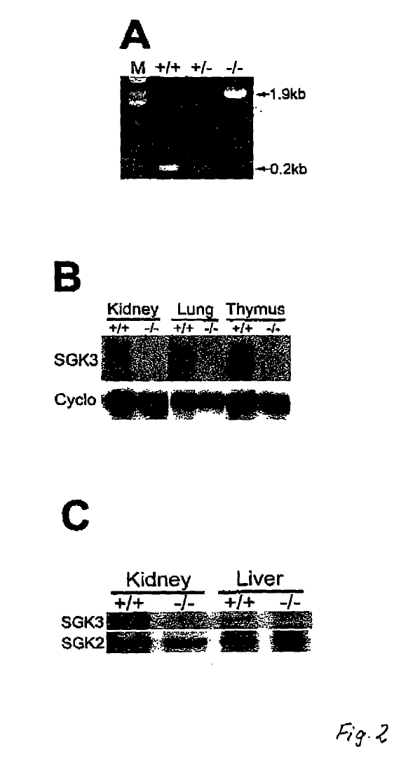

In this way, offspring from matings of sgk3^{+/-} mice were genotyped by PCR. The result of such an experiment is shown in FIG. 2A. Examination of 39 litters from sgk3^{+/-} showed a slight under-representation of sgk3 null mice (sgk3^{+/+} (27%), sgk3^{+/-} (51%) and sgk3^{-/-} (22%) mice)) with normal sex ratio. Both, male and female sgk null mice were fertile.

The predicted sizes of amplicons derived from the wild type allele are 0.2 kb, whereas those from the disrupted sgk3 allele are 1.9 kb. The detected sizes of amplicons therefore correspond to the predicted size, as this is to be seen in FIG. 2A.

### EXAMPLE 3

### Northern Blot Analysis of Mice RNA

Total RNA of the animals from a panel of tissues including kidney, liver and thymus was isolated using STAT-60 reagent (Tel-Test Inc.). 8 μg of RNA was resolved by formaldehyde-agarose gel electrophoresis, transferred to Hybond-NX membrane (Amersham Pharmacia), and probed with a fragment spanning the entire sgk3 open reading frame. The membrane was analyzed via autoradiographic exposure. After that, the membrane was stripped and reprobed for cyclophilin (Cyclo) as a loading control.

The results of such an experiment are shown in FIG. 2B. These data confirm the absence of SGK3 mRNA in homozygous mice. Contrary to that, these Northern blot analysis of tissues from twelve week old wild type mice revealed moderate to high expression levels of SGK3mRNA in kidney, lung and thymus (FIG. 2B) as well as in heart, liver and skeleton muscle with lower expression in adrenal gland, brain skin, spleen and fat. (data not shown).

### EXAMPLE 4

### Western Blot Analysis of Mice Proteins

Western blot analysis was performed as described in Chen, S. A. et al., Proc Natl Acad Sci USA (1999) 96, 2514-2519. Protein extracts from wild type and sgk3 null mice were used for Western blot analysis using an SGK antibody that cross-reacts with SGK2 and SGK3. To distinguish between the two SGK isoforms, SGK2 and SGK3 proteins synthesized in a coupled reticulocyte system (Promega) were analyzed an the same blot.

FIG. 2C shows Western blot analysis of protein extracts from kidney and liver from wild type (+/+) and homozygous (−/−) mice. The predicted size of SGK3 protein was observed in sgk3^{+/+} mice 56.4 kDa. The SGK2-specific band is shown to confirm equal loading. In accordance with the Northern blot results, these data show the absence of SGK3 protein in sgk3 null mice.

### EXAMPLE 5

### Visual Analysis of Changes in Hair Growth

Heterozygote (+/−) and sgk null (−/−) litter-mates were photographed at post-partum day 10 (P10; during hair follicle morphogenesis), at P28, and at seven months of age.

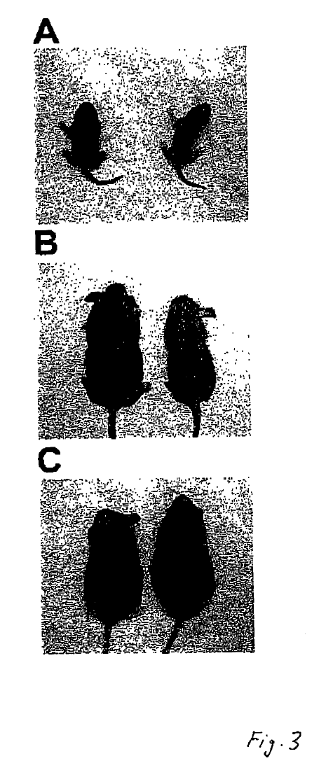

The result of such an experiment is presented in FIG. 3. The heterozygote littermates are shown on the left of each panel and the sgk3 null littermates are shown on the right of each panel. Photographs shown represent typical appearance at each stage for both sexes.
sgk3 null mice appeared normal at birth, but by P10 clearly displayed scarce hair growth relative to wild type littermates (FIG. 3A); heterozygotes were indistinguishable from wild type mice (not shown).

This initial abnormality persisted for at least four weeks (FIG. 3B); as the sgk3 null mice increased in age, the hair became increasingly thick (FIG. 3C) at all ages. sgk3 null mice displayed wavy coat fur and curly vibrissae.

### EXAMPLE 6

### Histological Studies on Mice, Immunohistochemistry and Tunel

To analyze skin morphology, dorsal skin was biopsied and fixed overnight in 10% neutral buffered formalin (Fisher). Samples were dehydrated, paraffin-embedded and sectioned (6 μm). For basic morphology, sections were deparaffinized and stained with hematoxylin and eosin. At 4 months, samples were obtained from male wild type and sgk3 null litter-mates. For other time-points, samples were taken from heterozygote and sgk3 null littermates to obtain sufficient numbers.

In mice, hair follicle development (also called morphogenesis) begins at embryonic day 14.5 (E14.5) with placode formation, and is completed at P16 with termination of the first growth phase (first anagen).

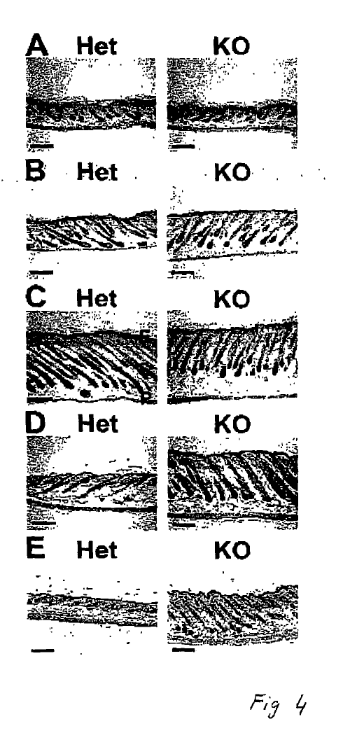

To determine at what stage hair morphogenesis becomes abnormal, dorsal skin was harvested from (FIG. 4A) P1, (B) P4, (C) P5, (D) P19, (E) P22 sgk3 null mice (KO) and heterozygotes (Het) littermates. Paraffin-embedded skin sections (6 μm) were prepared, deparaffinized, stained with hematoxylin and eosin and examined by lightmicroscopy, as described above. In FIG. 4 a representative section from each time-point and genotype is shown. The scale bar represents 200 μm.

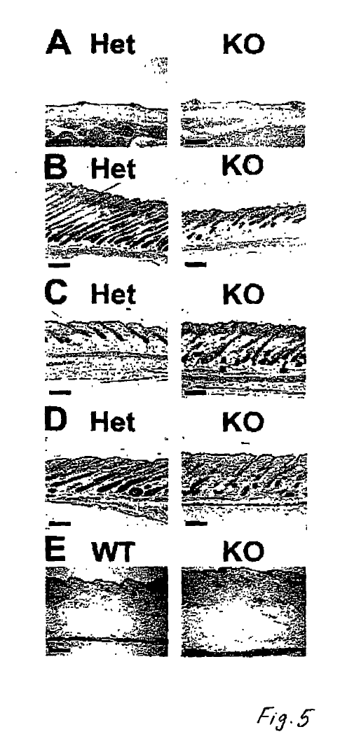

In a parallel study, whose results are presented in FIG. 5, (A) E15.5 embryos or dorsal skin from (B) P16, (C) P26, (D) P30, (Het) and sgk3 null (KO) littermates, and (E) four months old wild type (WT) and sgk3 null (KO) litter-mates were harvested, sections were prepared, deparaffinized, stained and examined as aforesaid. The scale bar in this figure also represents 200 μm.

At all time-points examined, the hair follicles (dark stained oblong structures) of heterozygote mice underwent appropriately-timed cycling, and were indistinguishable from wild type mice (FIGS. 4A to E and 5). sgk3 null embryos displayed normal induction of hair follicle morphogenesis, as indicated by the presence of placodes and germs at E15.5 (FIG. 5A), and development appeared normal at P1 (FIG. 4A). There was also no clear consistent difference between genotypes at P3, with considerable variability in morphology within genotypes (now shown).

However, by P4, there was an emerging defect in morphogenesis, characterized by a failure of the hair bulb to enlarge and migrated deep into the subcutis (FIG. 4B). This defect became more pronounced by P5 (FIG. 4C). There was no difference in thickness of epidermis, dermis or subcutis, revealing an intrinsic defect in the hair follicle.

The defect persisted through follicle morphogenesis (i.e. at P7, P10 and P14, not shown). By P16, heterozygotes displayed large numbers of anagen hair follicles residing deep in the subcutis, characteristic of late hair follicle morphogenesis (FIG. 5B).

By P19, heterozygotes were in catagen, characterized by the appearance of apoptotic Tunel positive cells in the hair bulb (FIG. 4D), followed by hair bulb involution and a reduction in hair follicle length. By P22, they had completed the first hair cycle and were in telogen (FIG. 4E); characterized by thinning of the subcutis, and the entire follicle residing in the dermis and lacking an inner root sheath.

The second anagen phase had begun by P26, and was advanced by P30 (FIGS. 5C and D). The dermal papilla became enlarged, the hair bulb reformed, the inner root sheath formed and a new hair shaft began to develop.

In contrast, the follicles of sgk3 null mice displayed the same abnormality apparent at P5 at all these time-points (FIGS. 4D and E, and 5B to D), suggesting a delayed or aborted progression through the initial hair cycle. Morphologically, the developing hair follicles in sgk3 null mice were disorganized, lacking the uniform orientation observed in wild type and heterozygote mice. The early hair abnormality in sgk3 null mice, thus, appears to be a combination of impaired follicle progression through the first hair cycle and abnormal follicle organization. Furthermore, there was also an apparent expansion of the outer root sheath of the hair follicle. Analysis of dorsal skin samples taken from mice aged four months revealed that sgk3 null mice showed a distribution of follicles between anagen, catagen, and telogen. In wild type mice the majority of follicles were in a telogen (FIG. 5E).

These data suggest that there is an initial defect in the hair cycle, but follicles of sgk3 null mice are able to progress through the cycle.

Histological analysis in parallel studies revealed no gross abnormalities in any other tissues examined (data not shown).

### EXAMPLE 7

### Weight Analysis of Mice

It has been suggested that since SGK3 was identified as a factor in IL-3-mediated survival of lymphocytes, and shares activition pathways and substrate specifity with Akt, it might display functional overlap with Akt family members; cf. Virbasius, J. V. et al., Proc Natl Acad Sci USA (2001) 98, 1290B-12913. Akt null mice weigh 20-25 percent less than wild type mice from birth to at lease 14 moths of age; cf. Chen, W. S. et al., Genes Dev (2001) 15, 2203-2208; Cho, H. et al., J Biol Chem (2001) 276, 38349-38352.

To check whether sgk3 null mice (KO) also show less body weight compared to wild type mice, new born mice from 7 litters were weighed within 18 h of birth, sacrificed, and genomic DNA isolated from tails for genotyping and sexing by PCR as described.

The result of such an assay is presented in the following table.

| | | | WT | KO |
|---|---|---|---|---|
| | WT MALE | KO MALE | FEMALE | FEMALE |
| MEAN | 1.51 | 1.38 | 1.49 | 1.42 |
| n | 10 | 7 | 9 | 4 |
| SD | 0.15 | 0.13 | 0.15 | 0.03 |
| p value WTvKO | | 0.08 | | 0.38 |
| p value MvF | | 0.74 | | 0.54 |

The data from this table show that in contrast to akt2 null mice, sgk3 null mice did not differ at birth from wild type or heterozygotes littermates. However, by P10, sgk3 null mice (identified by fur appearance), weighed 8 percent less than wild type heterozygotes littermates (p=0.002, not shown).

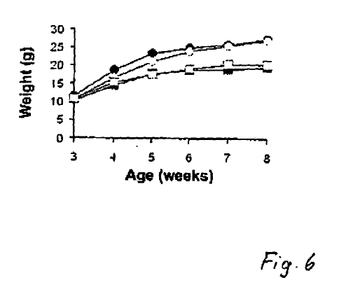

In order to determine, how the growth disturbance develops over the time male and female mice from 15 litters were weighed at weekly intervals from three to eight weeks of age. The results are shown in FIG. 6.

Data represent means at each age +/− S.E. (closed circles, male wild type; op en circles, male homozygotes; closed squares, female wild type; open squares, female homozygotes); n=10-17. The data were analyzed using the stat-view 4.5 software package.

Repeated measures ANOVA revealed significant differences between the growth rates of both, male and female sgk3 null mice relative to wild type: male sgk3 null mice showed a significantly reduced growth rate relative to wild type males (p<0.001). This difference was evident from 3 to 6 weeks of age, but disappeared by 7 weeks of age. In contrast, female sgk3 null mice grew more rapidly than wild type female mice over the same time-course (p=0.02).

### EXAMPLE 8

### Glucose metabolism

akt1 null mice have normal glucose metabolism. akt2 null mice, however, display insulin resistance and a diabetic phenotype. In order to determine whether sgk3 null mice display a disordered glucose metabolism, wild type and sgk3 null litter mates of the same sex were fastened over night (16 h) and then injected intraperetoneally with 1 mg/g of body weight D-glucose (10% (w/v) stock solution in phosphate buffered saline). Blood samples were collected from the transversely sectioned tip of the tail and whole blood glucose measured using a Glucometer Elite (Bayer) at 0 min (just before glucose injection), and at 15-, 30-, 60-, 90-, 120-, 180- and 240-min-intervals after the glucose load.

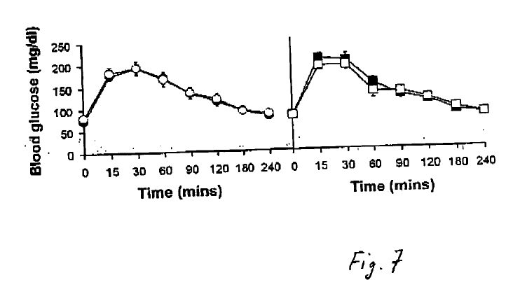

The result of this experiment is shown in FIG. 7. Data are represented as mean ±SG (closed circles, male wild type; open circles, male homozygotes; closed squares, female wild type; open squares, female homozygotes); n=10 mice for each genotype and sex.

The glucose tolerance in 8-10 week old sgk3 null mice was indistinguishable from that of wild type mice in both sexes. sgk3 null mice display a normal glucose homeostasis. Hence, it appears that there is either redundancy amongst SGK/Akt isoforms in the control of glucose homeostasis, or that SGK3 is not required for normal glucose metabolism.

As a result the mild transient growth abnormity of sgk3 null mice, however, may reflect partially overlapping functions of SGK3 and Akt1.

To summarize, experiments performed by the inventors substantiate a key position of SGK3 in the regulation and controlling of the hair growth cycle in mammalians. The modulation of the activity of SGK3 is in direct correlation to the hair growth of the mammalia and will therefore be an auspicious approach for the treatment of hair growth disorders.

1. A method for identifying a hair growth modulating substance, comprising:
(a) contacting a peptide derived from SGK3 and a test substance suspected to modulate hair growth, under conditions allowing the binding of said test substance to said peptide, and
(b) determining, whether said test substance modulates an activity of said peptide.

2. The method of claim 1, wherein said activity of said peptide is a kinase activity of said peptide.

3. The method of claim 2, wherein said test substance inhibits the kinase activity of said peptide.

4. The method of claim 2, wherein said test substance stimulates the kinase activity of said peptide.

5. A method for preparing a pharmaceutical/cosmetic composition for a treatment of a hair growth disorder, comprising:
(a) providing a hair growth modulating substance identified by the method of claim 1, and
(b) formulating said substance into a pharmaceutically/cosmetically acceptable carrier.

6. The method of claim 5, wherein said hair growth disorder is selected from the group consisting of: hair loss, baldness, unwanted hair growth, hypertrichosis, hirsutism, alopecia androgenetica, alopecia areata, alopecia areata universalis, and alopecia atrophicans.

7. The method of claim 6, wherein said hair loss is induced by chemotherapy.

8. The method of claim 6, wherein said baldness is a male pattern baldness.

9. A method for treating a human being affected by a hair growth disorder, comprising administering a hair growth modulating substance identified by the method of claim 1.

10. The method of claim 9, wherein said hair growth disorder is selected from the group consisting of: hair loss; baldness; unwanted hair growth, hypertrichosis, hirsutism; alopecia androgenetica; alopecia areata; alopecia areata universalis; and alopecia atrophicans.

11. The method of claim 10, wherein said hair loss is induced by chemotherapy.

12. The method of claim 10, wherein said baldness is a pattern baldness.

13. A hair growth modulating substance identified by the method of claim 1.

14. The substance of claim 13 that inhibits the activity of said SGK3-derived peptide.

15. The substance of claim 13 that stimulates the activity of said SGK3-derived peptide.

16. A method for identifying hair growth modulating substance, comprising:
(a) providing a transgenic non-human sgk3^{-/-} animal,
(b) administering a test substance to said animal, and
(c) determining, whether said test substance modulates hair growth of said animal.

17. The method of claim 16, wherein said animal is a mouse.

18. The method of claim 16, wherein said administering is performed by applying the test substance onto the skin of the animal.

19. The method of claim 16, wherein said administering is performed by local injections around and in an affected area.

20. The method of claim 16, wherein said administering is performed by systemic dosage of the substance to the animal.

21. A method for preparing a pharmaceutical/cosmetic composition for treatment of hair growth disorders, comprising:
(a) providing a hair growth modulating substance according to the method of claim 16, and
(b) formulating said substance into a pharmaceutically/cosmetically acceptable carrier.

22. The method of claim 21, wherein said hair growth disorder is selected from the group consisting of: hair loss, baldness, unwanted hair growth, hypertrichosis, hirsutism, alopecia androgenetica, alopecia areata, alopecia areata universalis, and alopecia atrophicans.

23. The method of claim 22, wherein said hair loss is induced by chemotherapy.

24. The method of claim 22, wherein said baldness is a male pattern baldness.

25. A method for treating a human being affected by a hair growth disorder, comprising: administering hair growth modulating substance identified by the method of claim 16.

26. The method of claim 25, wherein said hair growth disorder is selected from the group consisting of: hair loss, preferably induced by chemotherapy; baldness, preferably male pattern baldness; hirsutism; unwanted hair growth, hypertrichosis, alopecia androgenetica; alopecia areata; alopecia areata universalis; and alopecia atrophicans.

27. The method of claim 26, wherein said hair loss is induced by chemotherapy.

28. The method of claim 26, wherein said baldness is a male pattern baldness.

29. A hair growth modulating substance identified by the method of claim 16.

30. The substance of claim 29 that inhibits the activity of said SGK3-derived peptide.

31. The substance of claim 29 that stimulates the activity of said SGK3-derived peptide.

32. A transgenic non-human sgk3^{-/-} animal for investigating hair growth disorders.

33. The animal of claim 32, wherein said animal is a mouse.

34. A method for treating a human being affected by a hair growth disorder, comprising:
(a) providing a genetic construct coding for an antisense-sgk3 probe and/or for an sgk3-RNAi and/or for a transdominant inhibitory SGK3, and
(b) introducing said construct into a human being by means of gene therapeutic methods.

35. The method of claim 34, wherein said hair growth disorder is selected from the group consisting of: hair loss, baldness, unwanted hair growth, hypertrichosis, hirsutism, alopecia androgenetica, alopecia areata, alopecia areata universalis, and alopecia atrophicans.

36. The method of claim 35, wherein said hair loss is induced by chemotherapy.

37. The method of claim 35, wherein said baldness is a male pattern baldness.

38. The method of claim 34, wherein said construct is selected from the group consisting of: naked DNA or cDNA, naked RNA, plasmid DNA, plasmid RNA, vector DNA, vector RNA, and a non-virulent/non-pathogenic virus.

39. A method for treating a human being affected by a hair growth disorder, comprising:
(a) providing a genetic construct comprising a region coding for sgk3-derived segment under control of a promoter, and
(b) introducing said construct into a human being by means of gene therapeutic methods for expression of SGK3.

40. The method of claim 39, wherein said promoter is an inducible promoter.

41. The method of claim 39, wherein said hair growth disorder is selected from the group consisting of: hair loss, baldness, unwanted hair growth, hypertrichosis, hirsutism, alopecia androgenetica, alopecia areata, alopecia areata universalis, and alopecia atrophicans.

42. The method of claim 41, wherein said hair loss is induced by chemotherapy.

43. The method of claim 41, wherein said baldness is a male pattern baldness.

44. The method of claim 39, wherein said construct is selected from the group consisting of: naked DNA or cDNA, naked RNA, plasmid DNA, plasmid RNA, vector DNA, vector RNA, non-virulent/non-pathogenic virus, and a transformed bacteria.

45. A method for preparing a pharmaceutical composition for treatment of hair growth disorders, comprising:
(a) providing a genetic construct coding for antisense-sgk3, and
(b) formulating said construct into a pharmaceutically/cosmetically acceptable carrier.

46. The method of claim 45, wherein said hair growth disorder is selected from the group consisting of: hair loss, baldness, hirsutism, unwanted hair growth, hypertrichosis, alopecia androgenetica, alopecia areata, alopecia areata universalis, and alopecia atrophicans.

47. The method of claim 46, wherein said hair loss is induced by chemotherapy.

48. The method of claim 46, wherein said baldness is a male pattern baldness.

49. A composition, comprising a substance which modulates the activity of SGK3.

50. The composition of claim 49, further comprising a pharmaceutically acceptable carrier.

51. The composition of claim 49, further comprising a cosmetically acceptable carrier.

52. A composition, comprising a substance which causes a modulation of the activity of an SGK3-derived peptide, said substance identified by the method of claim 1.

53. The composition of claim 52, further comprising a pharmaceutically acceptable carrier.

54. The composition of claim 52, further comprising a cosmetically acceptable carrier.

55. A composition, comprising a substance which causes a modulation of the activity of an SGK3-derived peptide, said substance identified by the method of claim 16.

56. The composition of claim 55, further comprising a pharmaceutically acceptable carrier.

57. The composition of claim 55, further comprising a cosmetically acceptable carrier.
